(19) United States
(12) Patent Application Publication
(10) Pub. No.: US 2026/0263124 A1
(43) Pub. Date: Sep. 10, 2026

(54) Nail with Internal Locking Member

(71) Applicant: RLFA Biomedical Ltd, Sandyford (IE)

(72) Inventor: Robert Flavin, Wicklow (IE)

(21) Appl. No.: 19/547,128

(22) Filed: Feb. 23, 2026

Related U.S. Application Data
(60) Provisional application No. 63/767,897, filed on Mar. 6, 2025.

(51) Int. Cl. A61B 17/72

(52) U.S. Cl. CPC A61B 17/725

(57) ABSTRACT

A nail with internal locking member is provided. The nail has a proximal portion and a plurality of proximal through openings in the proximal portion. the through openings extend in a generally helical pattern around a perimeter of the proximal portion. The locking member has a plurality of through passages formed therein.

### BACKGROUND OF THE INVENTION

### Field of the Invention

The invention relates to a nail for long bone repair.

### Description of the Related Art

Repair nails are inserted lengthwise into a long bone, such as a femur or a tibia to provide support during repair of the bone after a break.

It would be beneficial to provide a locking mechanism that prevents the nail from rotating or otherwise moving relative to the bone after insertion.

### SUMMARY OF THE INVENTION

This Summary is provided to introduce a selection of concepts in a simplified form that are further described below in the Detailed Description. This Summary is not intended to identify key features or essential features of the claimed subject matter, nor is it intended to be used to limit the scope of the claimed subject matter.

In one embodiment, the present invention is a nail with internal locking member. The nail has a proximal portion and a plurality of proximal through openings in the proximal portion. the through openings extend in a generally helical pattern around a perimeter of the proximal portion. The locking member has a plurality of through passages formed therein.

### BRIEF DESCRIPTION OF THE DRAWINGS

The accompanying drawings, which are incorporated herein and constitute part of this specification, illustrate the presently preferred embodiments of the invention, and, together with the general description given above and the detailed description given below, serve to explain the features of the invention. In the drawings:

FIG. 1 is a side elevational view of a nail according to an exemplary embodiment of the present invention;

FIG. 2 is a perspective view of a locking member used with the nail of FIG. 1;

FIG. 3 is an end elevational view of the locking member of FIG. 2 inserted into the nail of FIG. 1;

FIG. 4 is a sectional view of the locking member of FIG. 2, taken along lines 4-4 of FIG. 2;

FIG. 5 is an end elevational view of a through a passage in the locking member of FIG. 2;

FIG. 6 is a sectional view of the nail of FIG. 1 with the locking member in an unlocked position;

FIG. 7 is a sectional view of the nail of FIG. 1 with the locking member in a locked position;

FIG. 8 is a perspective view of a distal end of a nail according to an alternative exemplary embodiment of the present invention;

FIG. 9 is a locking member used with the nail of FIG. 8;

FIG. 10 is a perspective view of an alternative embodiment of a locking member with a threaded through passage; and

FIG. 11 is a perspective view of another alternative embodiment of a locking member with a nylon ring inserted into a threaded through passage.

### DETAILED DESCRIPTION

In the drawings, like numerals indicate like elements throughout. Certain terminology is used herein for convenience only and is not to be taken as a limitation on the present invention. The terminology includes the words specifically mentioned, derivatives thereof and words of similar import. The embodiments illustrated below are not intended to be exhaustive or to limit the invention to the precise form disclosed. These embodiments are chosen and described to best explain the principle of the invention and its application and practical use and to enable others skilled in the art to best utilize the invention.

Reference herein to “one embodiment” or “an embodiment” means that a particular feature, structure, or characteristic described in connection with the embodiment can be included in at least one embodiment of the invention. The appearances of the phrase “in one embodiment” in various places in the specification are not necessarily all referring to the same embodiment, nor are separate or alternative embodiments necessarily mutually exclusive of other embodiments. The same applies to the term “implementation.”

As used in this application, the word “exemplary” is used herein to mean serving as an example, instance, or illustration. Any aspect or design described herein as “exemplary” is not necessarily to be construed as preferred or advantageous over other aspects or designs. Rather, use of the word exemplary is intended to present concepts in a concrete fashion.

The word “about” is used herein to include a value of +/−10 percent of the numerical value modified by the word “about” and the word “generally” is used herein to mean “without regard to particulars or exceptions.”

Additionally, the term “or” is intended to mean an inclusive “or” rather than an exclusive “or”. That is, unless specified otherwise, or clear from context, “X employs A or B” is intended to mean any of the natural inclusive permutations. That is, if X employs A; X employs B; or X employs both A and B, then “X employs A or B” is satisfied under any of the foregoing instances. In addition, the articles “a” and “an” as used in this application and the appended claims should generally be construed to mean “one or more” unless specified otherwise or clear from context to be directed to a singular form.

Unless explicitly stated otherwise, each numerical value and range should be interpreted as being approximate as if the word “about” or “approximately” preceded the value of the value or range.

The use of figure numbers and/or figure reference labels in the claims is intended to identify one or more possible embodiments of the claimed subject matter in order to facilitate the interpretation of the claims. Such use is not to be construed as necessarily limiting the scope of those claims to the embodiments shown in the corresponding figures.

It should be understood that the steps of the exemplary methods set forth herein are not necessarily required to be performed in the order described, and the order of the steps of such methods should be understood to be merely exemplary. Likewise, additional steps may be included in such methods, and certain steps may be omitted or combined, in methods consistent with various embodiments of the present invention.

Although the elements in the following method claims, if any, are recited in a particular sequence with corresponding labeling, unless the claim recitations otherwise imply a particular sequence for implementing some or all of those elements, those elements are not necessarily intended to be limited to being implemented in that particular sequence.

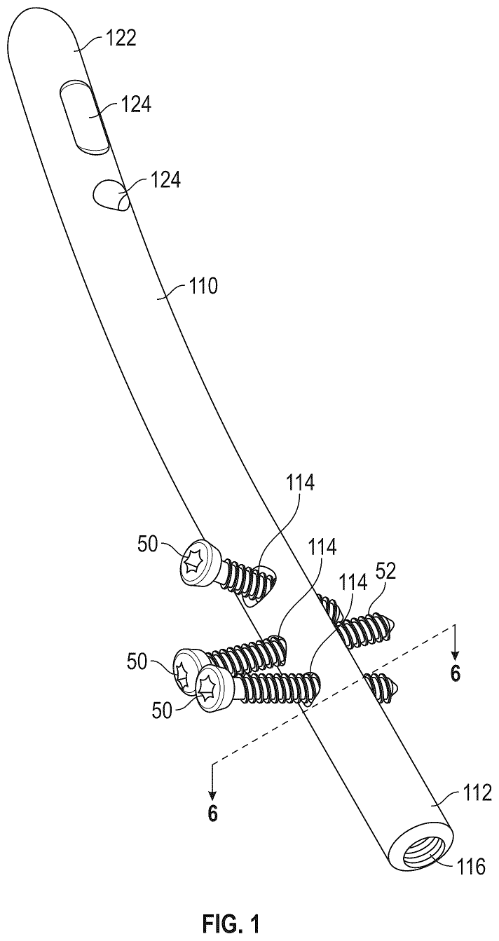

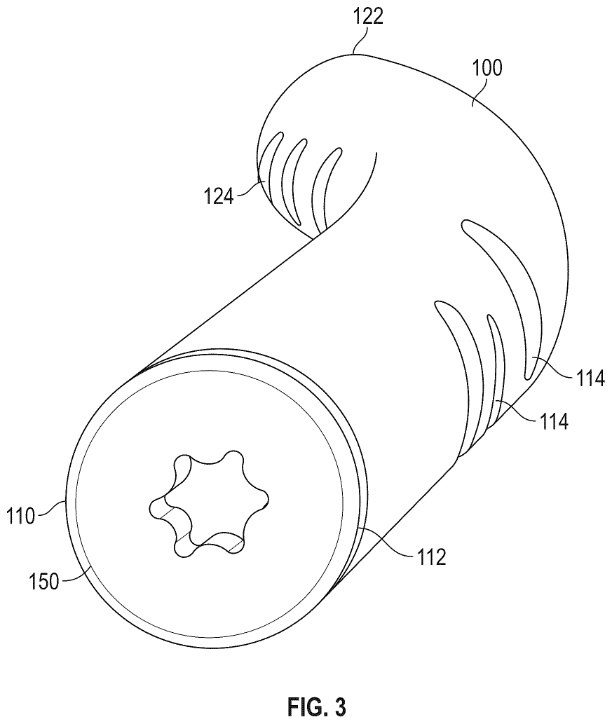

Referring to FIGS. 1-3, a nail assembly 100 according to a first exemplary embodiment of the invention is shown. Nail assembly 100 includes a nail 110 and a locking member 150. While nail 110 shown is a distal tibial nail, those skilled in the art will recognize that nail 110 can be other types of medical implant repair nail. By way of example only, nail 110 can be inserted lengthwise up a tibia (not shown) from the distal end of the tibia.

Nail 110 has a hollow proximal portion 112 and a plurality of proximal through openings 114. Through openings 114 can be located in a generally helical pattern around the perimeter of proximal portion 112. Proximal portion 112 of nail 110 is internally threaded with threads 116, typically past the distal-most proximal through opening 114. A distal portion 122 of nail 110 can also have a plurality of distal through openings 124. Distal portion 122 can be hollow or solid.

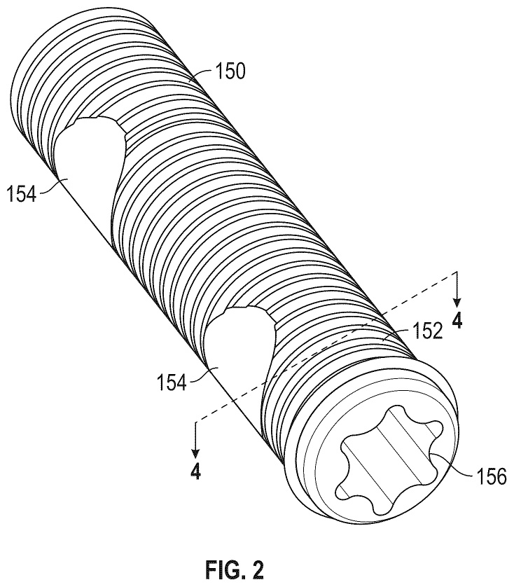

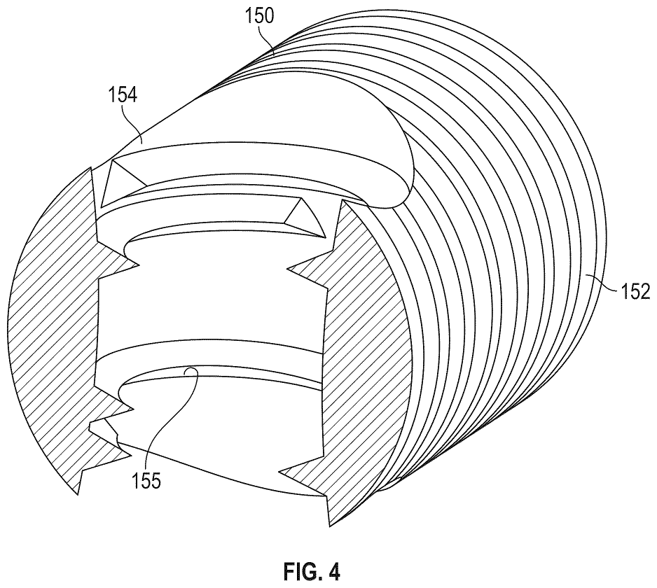

Locking member 150, shown in FIGS. 2-4, has external threads 152 that extend the length of locking member 150. Locking member 150 also includes a plurality of through passages 154. Through passages 154 can have internal threads 155 to engage screw 50 when screw 50 is inserted therethrough. A recessed head 156 at the proximal end of locking member 150 can accept a driver (not shown), such as a Torx® head wrench to thread locking member 150 into threads 116.

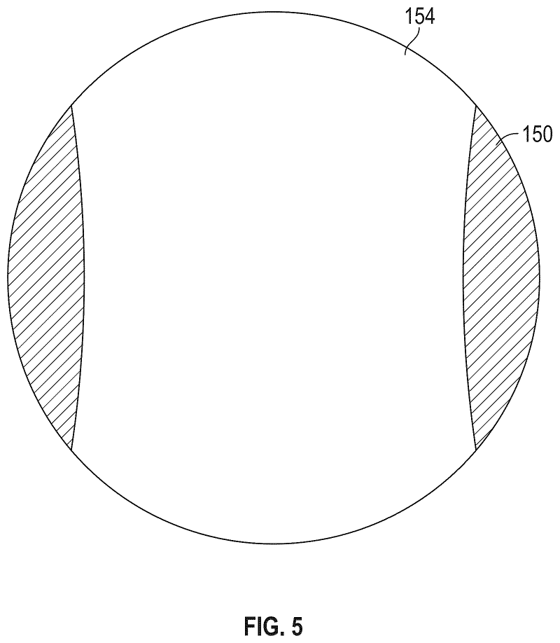

FIG. 5 shows an exemplary hourglass shaped through passage 154 in locking member 150. The hourglass design allows for the engagement of the sides of the through passage 154 into the screw threads 52 when locking member 150 is turned.

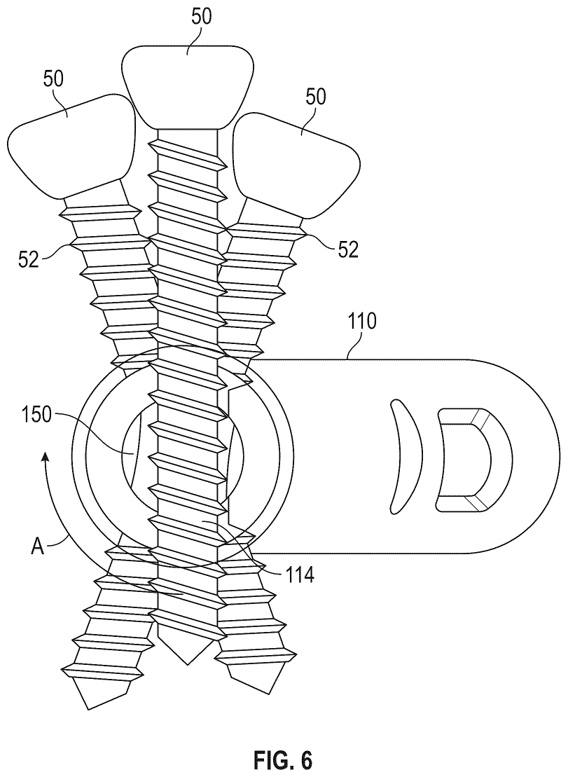

When locking member 150 is fully threaded into nail 110, through passages 154 align with through openings 114 so that screws 50, shown in FIG. 1, can be inserted through both through openings 114 and through passages 154 to secure nail 110 in place, as shown in FIG. 6.

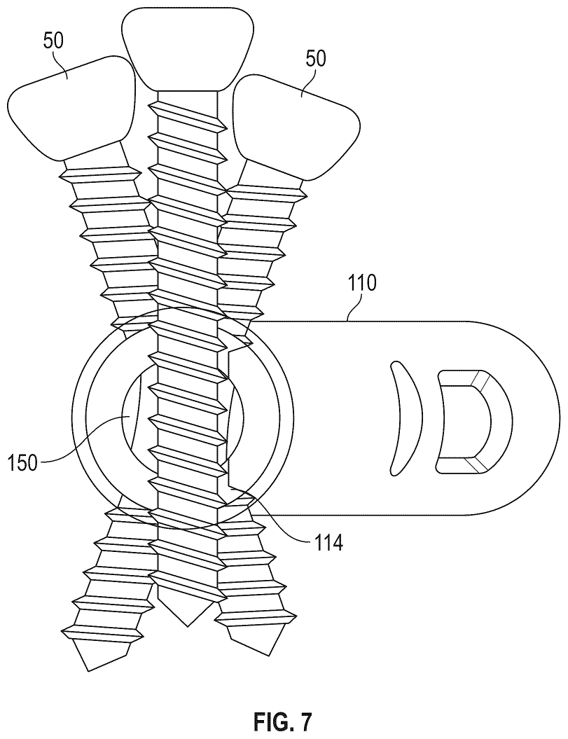

To further secure the screws 50 inside nail 110, locking member 150 is further tightened, in the direction of arrow “A” (FIG. 6) as shown in FIG. 7, wedging each screw 50 between a proximal side of through opening 114 and a distal side of through passage 154, preventing the screws 50 from inadvertently backing out.

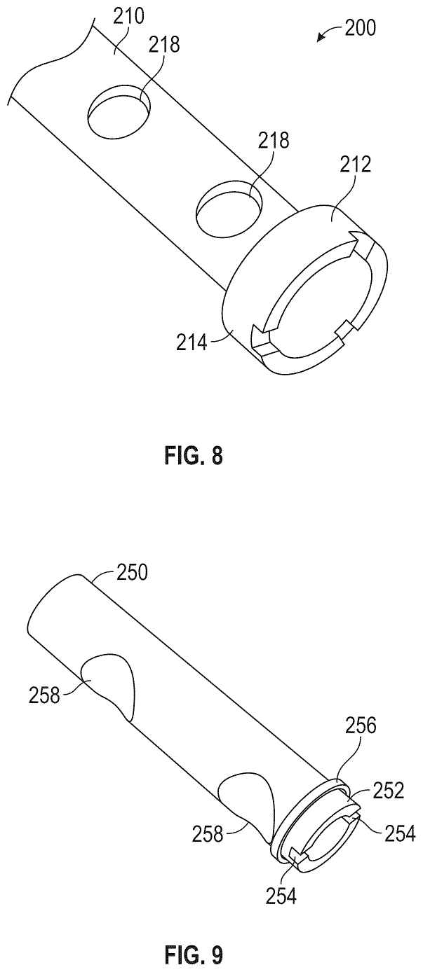

Referring now to FIGS. 8 and 9, a nail assembly 200 according to a second exemplary embodiment of the invention is shown. Nail assembly 200 includes a nail 210 and a locking member 250.

Nail 210 includes a cannulated proximal end 212 having a flange 214 surrounding proximal end 212. Proximal end 212 of nail 210, distal of flange 214, includes a plurality of through openings 218. In an exemplary embodiment, through openings 218 are formed in a helical pattern around nail 210.

Locking member 250 includes a proximal end 252 having a pair of diametrically opposed recesses 254 formed therein. Recesses 254 are used to accept a driver head (not shown) that is used to drive locking member into proximal end 212 of nail 210. A gasket, a nylon o-ring, or other sealing member 256 extends circumferentially around proximal end 252 of locking member 250. Sealing member 256 wedges locking member 250 into proximal end 212 of nail 210.

Locking member 250, distal of sealing member 256, includes a plurality of through openings 258. When locking member 250 is fully inserted into nail 210, through openings 258 align with through openings 218 so that securing screws (not shown) can be inserted through both through openings 258, 218 to secure nail 210 to bone. Locking member 250 can then be rotated in either direction, wedging each screw between the walls of through opening 218 and through opening 258, preventing the screws from inadvertently backing out.

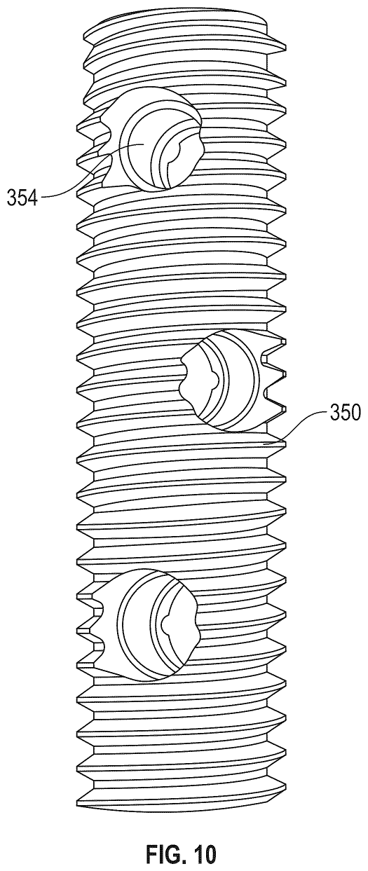

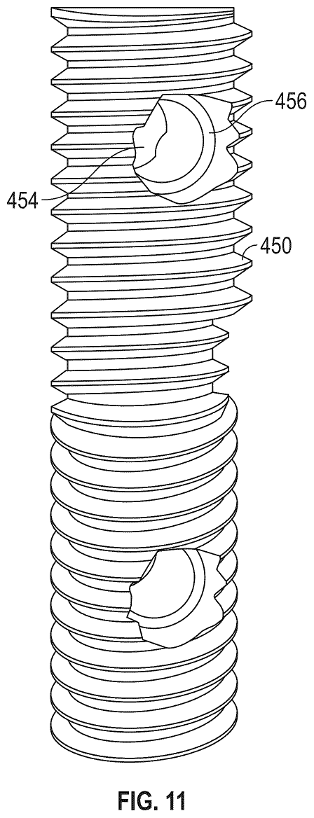

Referring now to FIGS. 10 and 11, instead of a smooth through passage 154, locking member 350 has a threaded through passage 354. Through passage 354 is provided without a nylon ring inserted into passage 354, whereas locking member 450 has a nylon ring 456 inserted into threaded through passage 454.

It will be further understood that various changes in the details, materials, and arrangements of the parts which have been described and illustrated in order to explain the nature of this invention may be made by those skilled in the art without departing from the scope of the invention as expressed in the following claims.

I claim:

1. A nail with internal locking member comprising:
a nail having:
a proximal portion; and
a plurality of proximal through openings in the proximal portion; and
a locking member having a plurality of through passages formed therein.

2. The nail with internal locking member according to claim 1, wherein the nail comprises a distal tibial nail.

3. The nail with internal locking member according to claim 1, wherein the proximal portion of the nail is hollow.

4. The nail with internal locking member according to claim 1, wherein the through openings extend in a generally helical pattern around a perimeter of the proximal portion.

5. The nail with internal locking member according to claim 1, wherein the proximal portion of the nail is internally threaded.

6. The nail with internal locking member according to claim 5, wherein the internal threads extend past a distal-most proximal through opening.

7. The nail with internal locking member according to claim 1, wherein the nail further comprises a distal portion, the distal portion having a plurality of distal through openings.

8. The nail with internal locking member according to claim 7, wherein the distal portion is hollow.

9. The nail with internal locking member according to claim 7, wherein the distal portion is solid.

10. The nail with internal locking member according to claim 1, further comprising a flange surrounding the proximal portion.

11. The nail with internal locking member according to claim 10, wherein the proximal portion, distal of the flange, includes a plurality of through openings.

12. The nail with internal locking member according to claim 1, wherein the through openings are formed in a helical pattern around the nail.

13. The nail with internal locking member according to claim 1, wherein the locking member includes a proximal end having a pair of diametrically opposed recesses formed therein.

14. The nail with internal locking member according to claim 13, wherein a sealing member extends circumferentially around the proximal end of the locking member.

15. The nail with internal locking member according to claim 14, wherein the sealing member wedges the locking member into the proximal end of the nail.

16. The nail with internal locking member according to claim 14, wherein the locking member, distal of the sealing member, includes a plurality of through openings.

17. The nail with internal locking member according to claim 1, wherein the locking member comprises external threads that extend the length of the locking member.

18. The nail with internal locking member according to claim 1, wherein the through passages have internal threads.

19. The nail with internal locking member according to claim 1, wherein each of the through passages has an hourglass shape.

20. The nail with internal locking member according to claim 1, such that, when the locking member is fully threaded into the nail, one of the through passages aligns with one of the through openings.
